(19) United States
(12) Patent Application Publication
(10) Pub. No.: US 2013/0022942 A1
(43) Pub. Date: Jan. 24, 2013

(54) SELF-OSTEOTOMIZING AND GRAFTING BONE IMPLANT

(71) Applicant: Parsa T. Zadeh, Beverly Hills, CA (US)

(72) Inventor: Parsa T. Zadeh, Beverly Hills, CA (US)

(21) Appl. No.: 13/553,678

(22) Filed: Jul. 19, 2012

Related U.S. Application Data
(60) Provisional application No. 61/510,009, filed on Jul. 20, 2011.

(51) Int. Cl. A61B 17/86; A61C 8/00

(52) U.S. Cl. 433/174; 606/301; 606/312; 433/173

(57) ABSTRACT

A bone implant includes a head and a core body extending from the head to a tip. Multiple osteotomy blades extend outwardly from at least a portion of the core body, arranged end-to-end to form a spiral thread. The implant, and particularly the osteotomy blades, are configured to self-osteotomize and channel cut bone between the blades and facilitate bone growth and grafting and integration of the implant to the bone.

### BACKGROUND OF THE INVENTION

The present invention generally relates to bone implants, such as dental implants. More particularly, the present invention is directed to a self-osteotomizing and self-grafting bone implant which creates its own osteotomy and facilitates bone growth and integration of the implant.

Traditionally, orthopedic medicine and dentistry have copied older established industries, like carpenters, to create fasteners for prosthetic items to be attached directly to bone in the form of various cone screws. In such non-medical, inanimate industries, as in cases of wood, plastic or metal, the principal of direct fasteners is based upon compressibility (wood), flexibility (plastic) or malleability (metal) or a combination of these properties being fastened to. In all these cases, a hole is created in the receiving material slightly smaller than the selected screw or fastener for the job.

The material shavings from these drillings have no cohesive or adhesive properties and are removed from the drilling site by the spiral action of the drill and discarded. The mass of the material that is removed by the drill is replaced mainly by the body of the screw or fastener. The threads of the fastener take advantage of the three properties of compressibility, flexibility and malleability of the receiving material to engage it with large enough frictional force so as to secure the fastener to the recipient material. The ultimate tightness or securement of the fastener in non-vital objects is the same initial tightness that is achieved by the frictional forces between the body of the screw and the walls of the hole and engagement of the threads into the material. Such non-vital structures (wood, plastic, metal) are usually homogonous in nature with predictable compressibility, flexibility or malleability factors and therefore the strength and behavior of the fastener can be controlled by the various properties of the fastener body and threads.

Human bones, however, have different properties depending on their location. Each bone has different properties from outside to inside. Hip bone, spines and upper jaw are porous, whereas the lower jaw, cranium and long bones are impervious at the outer shell. They all have spongy and softer structure as their core is approached. This diverse structure of the bones from one part of the body to another and within the same area from cortex (outer layer) to medulla (inner layer), makes the bone an unpredictable material for implants and fasteners. Inconsistencies in vital bone structure have resulted in many limitations in the current procedures. This has resulted in medical professionals and medical device engineers establishing over engineering and rescuing techniques, such as placing more implants or fasteners than needed or using fasteners or implants which are wider or longer than necessary, to make their procedures as successful as possible.

Although human bones have no sensory innervations, the bones experience pain by the stretch receptors in the periosteom, the outer thin covering of the bone. Therefore, while the drilling of the bone does not contribute to post-operative pain, placement of current bone screws or implants that rely on frictional forces for their stability cause expansion of the recipient bone, resulting in the main source of post-operative pain in orthopedic and dental implant surgeries.

The limitations and unpredictable bone qualities are many times greater in dental implant surgery as the implants are placed in place of freshly extracted teeth or teeth that were previously lost, such as due to chronic infections that created voids in the bone. In current dental implant systems, the relative condensability of the bone is taken advantage of for initial implant stability. For implants supporting dental restorations, a hole (slightly smaller in diameter than that of the proposed implant) is made in the bone (an osteotomy) by drilling at 800-1500 rotations per minute (RPM), typically with the use of saline coolant. The process usually involves creating progressively larger diameter holes which are drilled into the jawbone. Special twist drills are used in increasing the diameter until a hole of a size of 0.2-0.4 mm smaller than the implant cylinder or body is achieved.

The implant is then either tapped into this hole or more commonly “screwed” into the hole, much like a screw is driven into wood. Depending on the density of the recipient bone and the implant system in use, the osteotomy (hole) may be tapped before implant placement or the implants come with self-tapping features. In all these cases, the space for the implant is created mostly by drilling the native bone out and the implant is initially stabilized by condensing the immediate adjacent bone due to the implant being slightly larger than the tapped hole or osteotomy.

Creating a perfectly sized and shaped osteotomy is the greatest challenge for the implant dentist. Taking into consideration the fact that this osteotomy is performed in a physically unpredictable bone mass in the oral cavity between tongue and cheek, in a wet and bloody field with potential operator hand movement and patient movement creates many challenges for successful implant placement. Physically, jawbone in a live person varies greatly and unpredictably in density, condensability, texture and hardness from one site to another and at the same site from one mm in diameter or depth spot to the next. Live human bone is erratically fragile in small thicknesses. This fragility particularly complicates osteotomy creation in multi-rooted teeth sockets where thin webs of bone are the only anatomically correct position for the implant. All of these factors further depend on the condition and time of the extracted tooth and age of the implant recipient.

In current systems, the sequential drilling protocol removes and brings to surface any native bone that has occupied the space of the future implant. The bone shavings are often suctioned away along with the coolant liquid. Although there are commercially available “bone traps” that can be used to trap these shavings by the surgical suction mechanism, there are concerns with harvesting the bone in this manner due to potential bacterial contamination. Moreover, due to the nature of the suction mechanism, the trapped bone is repeatedly and cyclically washed and dried in the trap before it is recovered, thereby compromising the vitality and viability of the removed bone.

It can take a period of approximately three to six months after the emplacement of the body portion of the implant within the osteotomy for bone tissue to grow into the surface irregularities of the implant and secure the body portion of the implant in place within the bone bore or osteotomy. Following this three- to six-month period, an artificial tooth or other prosthetic component is typically secured to the implanted body portion. The most common cause of implant failure is the lack of initial stability, which is nothing but the inability and limitations of the system to create the perfectly sized and shaped osteotomy for the chosen implant and patient. It is important to know that the perfect size of the osteotomy for each implant size varies and depends on the condensability of the bone in that site, which can only be accurately known while the implant is being seated in the osteotomy. Inappropriate osteotomy size for a particular site is the most common cause of implant waste at dental offices that contributes to unnecessary higher cost to the consumers.

If the osteotomy size was overestimated, the primary stability suffers with risk of early mobility and implant loss in one to two weeks. If the size was underestimated, the primary stability will be excellent, but the excessive pressure at the implant bone interface, either through ischemic necrosis of the bone layer adjacent to the implant or through enzymatic activity from the pressure, the implant fails in three to four weeks.

Another reason for bone necrosis and subsequent failure of dental implants is damaging the osteotomy site by overheating it during drilling. An overused worn drill in a hard bone can generate enough heat to damage the bone to the extent that the implant does not integrate. Most implant systems recommend frequent changing of the drill sets, and others recommend “single use” drill sets to ensure sharp cutting edges every time. Needless to say, either way, there is a high per-implant cost in drilling supplies associated with the current systems.

In places where the implant is placed in thin bones, like the septum of a multi-rooted tooth, the success of current implants is limited due to the high chance of fracture of this septum either by sequential drillings or by the pressure of the implant itself.

The success of osseo-integration depends on microscopically close adaptation of the vital host bone to the implant surface. The immediately placed implant by virtue of the way that it has become to be in its final position, such as by rotation, although immobile by at least a tripod of tight areas, has gaps filled with blood in its bone-implant interface. Provided that the conditions are favorable, this implant is considered “oseo-integrated” when new bone cells grow into these gaps, totally obliterating any space between the host bone and the implant. This process takes approximately two to six months and hence the typical waiting period of three to six months following implant placements for integration. If part of the implant surface is in grafted bone, other than autogenous bone, the integration time is further extended because usually the grafted material has to first get resorbed and then host bone grows into its space. Any micro or macro movement of the implant surface during this period prevents formation of bone next to its surface and results in failure.

Accordingly, there is a continuing need for an improved bone implant which will consistently result in adequate and quick anchoring of the implant to the bone, and thus implant stability. The present invention fulfills these needs, and provides other related advantages.

### SUMMARY OF THE INVENTION

The present invention resides in a self-osteotomizing and self-grafting bone implant, with macro-stabilizing features, that osseointegrates within a much shorter time period. The implant generally comprises a head having a core body extending from the head to a tip. Multiple osteotomy blades extend outwardly from at least a portion of the core body and are arranged end-to-end forming a spiral thread.

An upper surface generally directed towards the head or a lower surface generally directed towards the tip of the osteotomy blades defines a depression adapted to channel bone material cut by the implant to a space between the upper and lower surfaces of adjacent osteotomy blades. Typically, the depression is generally V-shaped or U-shaped. The depression is defined by a first angled ramp of the surface of the osteotomy blade extending from the core body and a second angled ramp of the surface of the osteotomy blade extending from a peripheral outer surface of the osteotomy blade towards the first angled ramp.

At least a portion of the osteotomy blades have an enlarged generally flat peripheral outer surface in the form of a stabilizing wall generally facing away from the core body. The enlarged generally flat peripheral outer surface defines a cutting edge. The peripheral outer surface of the osteotomy blade typically has a generally triangular cross-section.

A plurality of apertures are formed through the osteotomy blades having a size and configuration permitting blood vessels to grow therein. Surfaces and edges formed by the apertures are generally non-cutting flat or rounded edges and surfaces. The apertures may comprise open-face apertures formed in peripheral surfaces of the osteotomy blades.

The implant is generally tapered from the head to the tip. The tip is typically rounded and has a diameter substantially matching the diameter of a pilot hole drilled into the bone. At least a portion of the osteotomy blades adjacent the core body are of increasing cross-sectional thickness from the head towards the tip.

The head of the implant, in a particularly preferred embodiment, comprises a dental implant head adapted to receive an abutment and restoration. The head includes a generally cylindrical neck adjacent the core body. The outer surface of the head between the neck and an upper head surface is generally concave.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is a side perspective view of a bone implant embodying the present invention;

FIG. 2 is a side elevational view of the bone implant of FIG. 1;

FIG. 3 is a bottom view taken generally along line 3-3 of FIG. 1;

FIG. 4 is a cross-sectional view of the bone implant taken generally along line 4-4 of FIG. 1;

FIG. 5 is a partial cross-sectional view taken generally along line 5-5 of FIG. 1;

FIG. 6 is an enlarged view of area “6” of FIG. 1;

FIG. 7 is an enlarged view of area “7” of FIG. 1;

FIG. 8 is a side perspective view of another bone implant embodying the present invention;

FIG. 9 is a cross-sectional view taken generally along line 9-9 of FIG. 8;

FIG. 10 is a perspective view of a bone implant embodying the present invention having a dental abutment attached thereto;

FIG. 11 is a cross-sectional view taken generally along line 11-11;

FIG. 12 is a cross-sectional and diagrammatic view of a prior art implant, with a portion thereof exposed;

FIG. 13 is a cross-sectional diagrammatic view illustrating bone tissue having a pilot hole drilled therein for receipt of an implant embodying the present invention;

FIG. 14 is a cross-sectional and diagrammatic view similar to FIG. 13, but illustrating the implant of the present invention submerged in bone and gum tissues;

FIG. 15 is an enlarged view of area “15” of FIG. 14, illustrating channeling of bone fragments, in accordance with the present invention;

FIG. 16 is a side elevational view of another bone implant embodying the present invention;

FIG. 17 is a side elevational view of yet another bone implant embodying the present invention; and

FIG. 18 is a cross-sectional view taken along line 18-18 of FIG. 17.

### DETAILED DESCRIPTION OF THE PREFERRED EMBODIMENTS

As shown in the accompanying drawings, for purposes of illustration, the present invention resides in a self-osteotomizing and grafting implant, generally referred to by the reference number 10. As will be more fully described herein, most of the osteotomy is achieved by the implant 10 itself as it is being driven into place. The bone shavings from the osteotomy are collected around the implant 10 as it is seated and act as autogenous bone graft, filling in the voids of the implant structure and implant bone surfaces. Moreover, the implant 10 has both vertical as well as lateral stabilization due to its design, responsible for the macro-osseointegration feature of the implant.

In existing implant designs, the bone next to the implant is condensed and crushed to achieve initial stability. In the prior art, during the initial osteotomy, the bone is brought out to the surface by the twist drills and suctioned away with the irrigating solution. Once the implant is inserted, any voids or vents within the implant space are filled with blood. The blood-filled space must first be vascularized and then osteoblastic activity must fill the volume of the void with solid bone in order for the voids to contribute to the stability of the body of the implant. Thus, it takes many weeks and months for the implant to osseointegrate.

The present implant design allows the osteotomy to be made in exact and precise dimension of the implant as it is being driven into place with the bone immediate to the implant surface remaining intact, vital and uncondensed, therefore remaining fully vascularized. Since the implant of the present invention creates its own osteotomy while it is driven into place, connective tissue is not interposed between the implant and the bone. With existing implants, as the initial stability is achieved by undersizing the osteotomy for the intended implant, due to large variations in recipient site bone quality, it is impossible to design a standardized drilling protocol for the osteotomy undersizing in all bone types, and micromotion often results from osteotomy-implant size mismatch, resulting in many early implant failures. However, as will be described more fully herein, the implant of the present invention creates the osteotomy at its own dimensions, irrespective of the bone quality around it. This provides for optimal initial stability required for osseointegration while avoiding over condensing and necrotizing the immediate bone next to the implant surface due to excessive lateral pressure from inserting the implant in an undersized osteotomy for that bone quality, as is done in the prior art. The precise approximation of vital and intact bone to the implant surface requires much less osteoblastic (bone formation) activity to take place for osseointegration to take place. Thus, the features and design of the implant of the present invention results in an efficient and rapid osseointegration. This is “macro osseointegration”, which is the more rapid and efficient osseointegration of the implant.

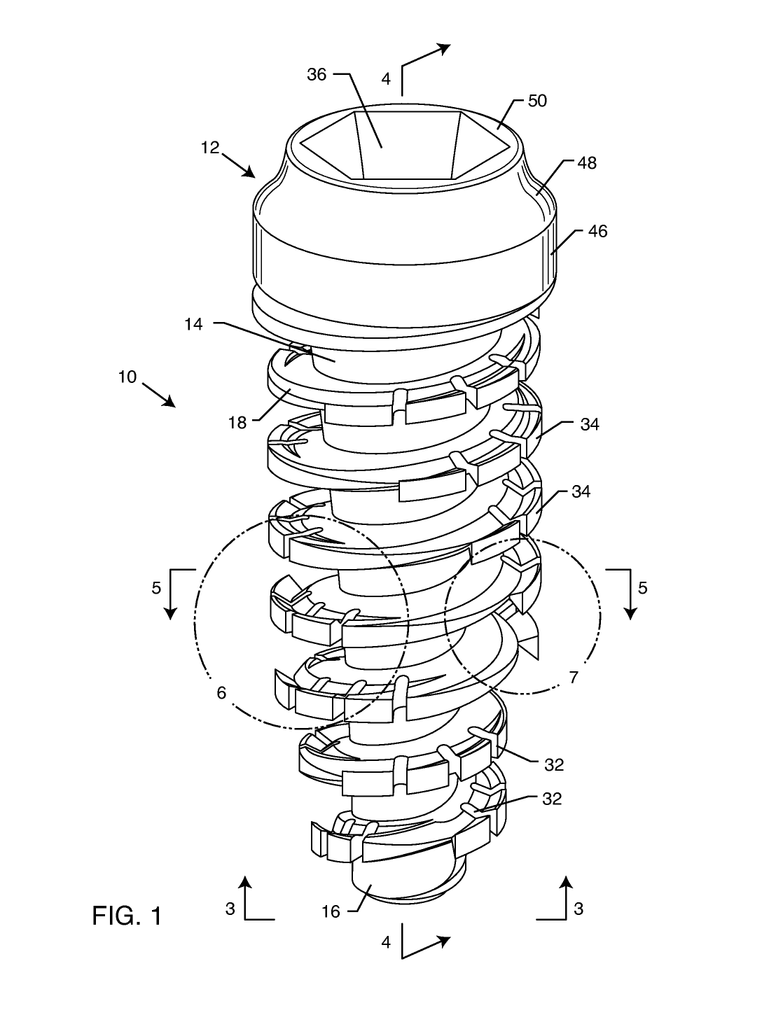

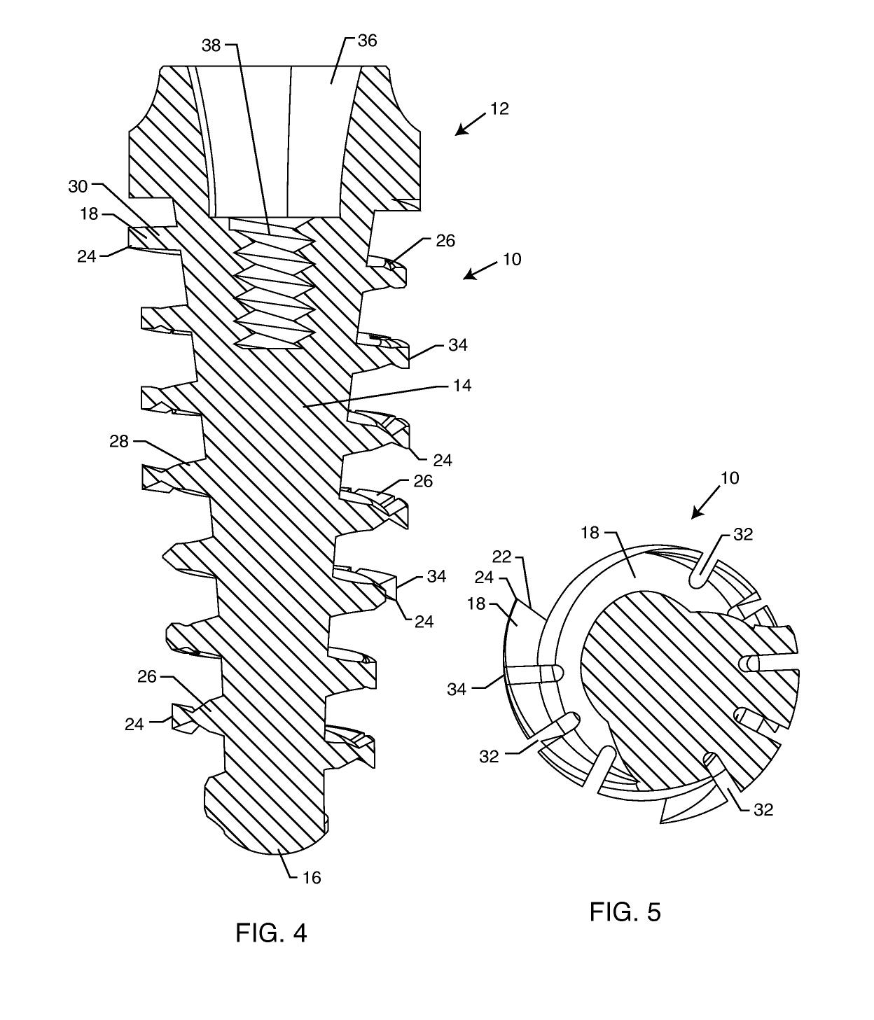

With reference now to FIGS. 1-7, the bone implant 10 generally comprises an upper head portion 12 having a core body 14 extending from the head to a tip 16 generally opposite the head 12. Typically, the core body 14 tapers from the head towards the tip 16. Moreover, the tip 16 is usually rounded, but it could be sharp for orthopedic applications. When inserting the bone implant of the present invention into a bone, a small pilot hole is usually formed having a diameter of approximately or slightly greater in size than the rounded tip 16 of the implant 10. The rounded tip 16 is non-cutting, but allows the implant 10 to follow the initial pilot hole and leads the implant in a predetermined direction dictated by the pilot hole.

A plurality of osteotomy blades extend outwardly from the core body 14. The osteotomy blades 18 are in essence arranged end-to-end, so as to form a spiral thread, as illustrated. It will be appreciated by those skilled in the art that the spiral thread may be continuous, but still formed of a plurality of osteotomy blades which are arranged end-to-end even if the osteotomy blades are not separated or distinct from one another other than their design and arrangement.

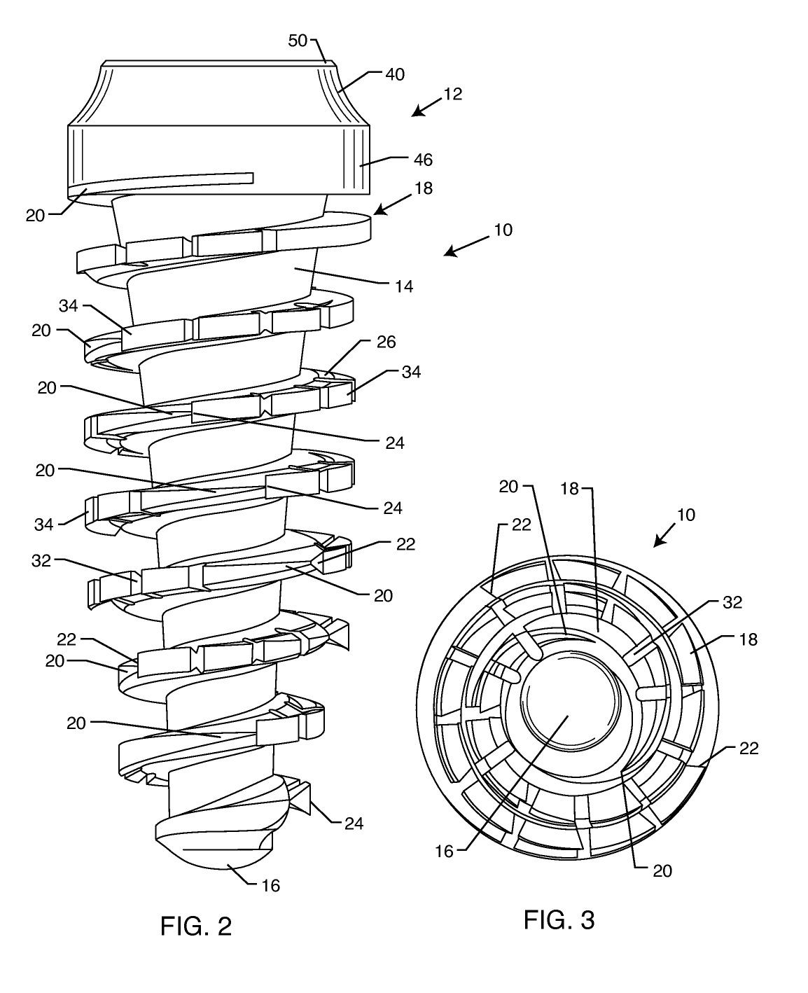

With specific reference to FIG. 2, in certain embodiments the osteotomy blades 18 extend all the way to the head portion 12 of the implant 10. Beginning portions of the blades 18 are referred to by the reference number 22, whereas the end portions are referred to by the reference number 20, so as to enable the reader to visualize the beginning and end of an osteotomy blade 18 as it extends and spirals around the core body 14 between the head 12 and tip 16 of the implant 10. Depending upon the application or manufacturing constraints or intended design, multiple blades 18 may form a single turn of the spiral thread, or a single blade 18 may form one or more turns of the spiral thread of the implant 10. The implant 10 may be designed and arranged such that the blades 18 closer to the head 12 are slightly larger than the blade 18 apical to it, or towards the tip 16. In this manner, the diameter of the blade 18 towards the head 12 is greater than that of the blade 18 adjacent to the tip 16, such that the overall implant 10 is tapered or conical in configuration.

In lieu of having threads, as is common in prior implants, the present invention incorporates osteotomy blades 18, which have bone-cutting peripheral edges 24. The cutting edges 24, and thus the blades 18, create the nearly exact space they will occupy in their lineal position as they get rotated or screwed into place in the bone. Typically, the cutting edges 24 face away from the core body 14.

The self-cutting nature of the bone cutting edges 24 of the osteotomy blades 18 provides more stability initially within the bone, in part due to the fact that the surrounding bone is not crushed or smeared in the placement process, as is the case when using current threaded implants. Due to this, there will be faster growth of bone, or osteointegration, around the blades 18 and implant 10.

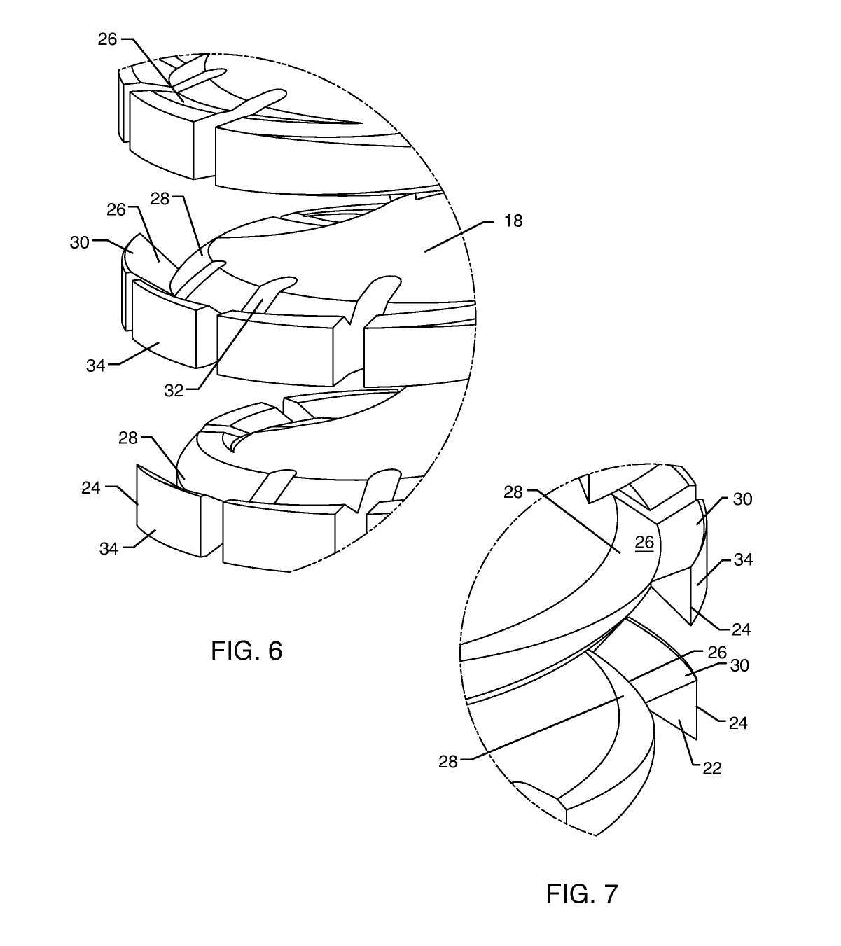

With reference now to FIGS. 4 and 6, in a particularly preferred embodiment, the thickness of the blades 18 and cutting edge 24 are greater towards the tip than the head 12. It will also be seen in FIG. 4 that the thickness of the blades 18 decreases from adjacent to the core body 14 towards their peripheral edges. This creates different thicknesses of cutting edges and blades, wherein the largest cutting edge and blade is equal to or slightly smaller than the size of the base of the smallest blade at that diameter.

With reference now to FIG. 6, at least a portion of an upper surface facing towards the head 12 and/or a lower surface facing generally towards the tip 16 have a depression 26 which serves to collect and channel bone shavings and other material cut by the implant 10 to a space between the upper and lower surfaces of adjacent osteotomy blades 18 and into spaces 32. As can be seen in FIG. 6, the depression is generally V-shaped or U-shaped, and defined by a first angled ramp 28 of the surface of the osteotomy blade 18 extending from the core body and a second angled ramp 30 on the surface of the osteotomy blade extending generally from the peripheral outer edge or surface of the osteotomy blade towards the first angled ramp 28, so as to define and form the depression or groove 26.

The depressions 26 are usually and generally concentric with the longitudinal axis of the core body 14, however, they can also be straight lines tangential with respect to the longitudinal axis of the core body due to manufacturing limitations as well. Nonetheless, depressions 26 are formed in the blades. The depressions 26 collect the bone shavings and condenses them as they are pushed towards the end of the depression 26, which is narrower than at the opening thereof.

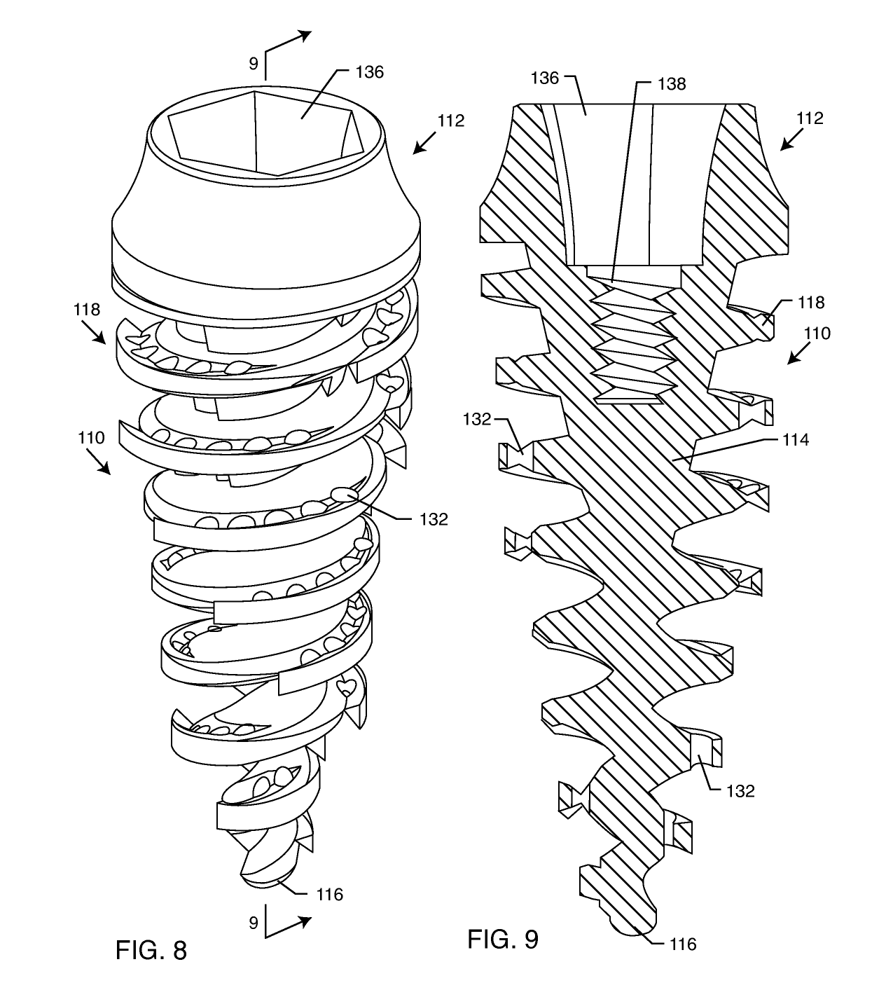

In a particularly preferred embodiment, apertures 32 are formed through the osteotomy blades 18. The apertures 32 are preferably of a size and configuration to permit blood vessels and bone to grow therein. As can be seen in the drawings, preferably the surfaces and edges formed by the apertures are generally flat or rounded so as to be non-cutting in nature so as to facilitate the growth of blood vessels therein. In the embodiment illustrated in FIGS. 1-7, the apertures are open-face and formed in the peripheral surfaces and edges of the osteotomy blades 18. Such open-face apertures are typically formed as a convenient manufacturing alternative to apertures formed completely within the osteotomy blades, such as illustrated in FIGS. 8-11. However, either arrangement will suffice provided that it facilitates growth of blood vessels and transfer of nutrients to the bone shavings and material cut by the osteotomy blades 18 during its placement and channeled by the depressions 26 towards the apertures 32 and between the adjacent osteotomy blades 18. These apertures allow blood vessels and bone to grow therein, resulting in macro-osseointegration of the implant.

Bone needs good blood supply for remodeling and healing. When the blades 18 of the implant 10 separate layers of bone from each other, circulation can suffer. This also occurs in current implant designs. The apertures 32 created through the blades 18 establish communication between layers of bone separated by the blades 18. The apertures 32, either in the form of the vertical open-faced slots or apertures along the periphery of the blades 18 or in the form of enclosed apertures 32 within the blades 18 of the implant, provide space for bone growth. This bone growth within the body of the implant provides for macro osseointegration and substantially adds to its early stability for loading, and allows efficient and rapid osseointegration of the implant.

Furthermore, the depressions 26 formed in the blades 18 serve to collect and lead the bone shavings and cut material towards the apertures 32. This can be particularly seen in FIG. 15. In accordance with the present invention, the osteotomy shavings (live host bone tissue) is guided by the strategically placed channels and grooves in the form of depressions 26 from the cutting edges of the blades 18 towards and into the apertures 32 of the implant body. Therefore, voids are filled with live bone tissue that can readily and rapidly heal together to provide rapid stability to the inserted implant.

It will be seen that the depressions 26 vary in size and depth across their length, thus serving to channel and guide the cut bone fragments and shavings as the implant 10 is screwed into position. The collection of the bone shavings into the depression 26 and between the adjacent blades 18, in conjunction with the apertures 32 formed in the blades 18 allows blood and bodily fluid flow therebetween. Eventually new blood vessels grow therein, and thus the bone shavings and cut bone material remain vital, and enhances the osteointegration of the implant 10 into the bone, creating a self-grafting feature of the implant 10. It is anticipated that the incorporation of the depressions 26, in conjunction with the apertures 32 and the self-osteotomizing blades 18, will cut the time it takes for solid bone to grow close to the implant surface and fill the voids, in order to osseointegrate, to less than half, and an anticipated three to six weeks only. Thus, it can be seen that the design of the implant of the present invention results in efficient and rapid osseointegration as compared to the prior art.

With reference now to FIGS. 1, 2, 4, 6 and 7, at least a portion, and typically the majority, of the osteotomy blades 18 have an enlarged generally flat peripheral outer surface defining a stabilizing wall 34. The stabilizing wall 34 faces generally away from the core body 14 and is generally parallel with the long axis of the implant. The generally flat peripheral outer surface defining the stabilizing wall 34 has a cutting edge 24 at its edge thereof. The incorporation of the stabilizing wall 34 at at least a portion of the outer peripheral surface of the blades 18 creates immediate lateral stabilization of the implant 10 within the bone.

With reference to FIGS. 4 and 7, the incorporation of the depressions 26 as well as the stabilizing outer peripheral wall 34 along a length of the osteotomy blade 18 creates a generally triangular cross-section along at least a portion of the outer peripheral portion of the osteotomy blades 18.

With reference now to FIGS. 8 and 9, an implant 110 very similar to that illustrated in FIGS. 1-7 is shown, with the only differences being that the apertures 132 are formed completely within the blades 118, instead of being formed as open-face apertures. These apertures 132 perform the same functions as described above with respect to apertures 32, in that they allow bodily fluid and blood flow and blood vessel generation to the bone shavings and cut material collected around them, as well as to the segments of bone which have been cut by the osteotomy blades. It will also be noted that the core body 114 of this embodiment, as illustrated in FIG. 9, is narrower. The width of dimension of the core body 114 may be adjusted according to the type of bone into which the implant 10 or 110 is to be placed. For example, harder or softer bone may require a larger or smaller core body 14 or 114, as dictated by the need for increased or decreased diameter osteotomy blades 18 or 118. Otherwise, the embodiment illustrated in FIGS. 8-11 has the same structure and function as that described above with respect to FIGS. 1-7. Thus, all of the reference numbers in FIGS. 8-11 that pertain to the implant 110 are increased by 100, for example the head is referred to by the reference number 112 and the tip 116, whereas the head and tip are referred to by the reference numbers 12 and 16, respectively, in FIGS. 1-7.

Aside from whether the apertures 32 or 132 are formed as open-face cut into the osteotomy blades 18 or formed completely in the osteotomy blades 18, the number of blades, thickness of each blade, and the spaces between adjacent blades can be determined by the physical properties of the metallic alloy chosen for the implant, manufacturing limitations, and physiological requirements of implanted bone.

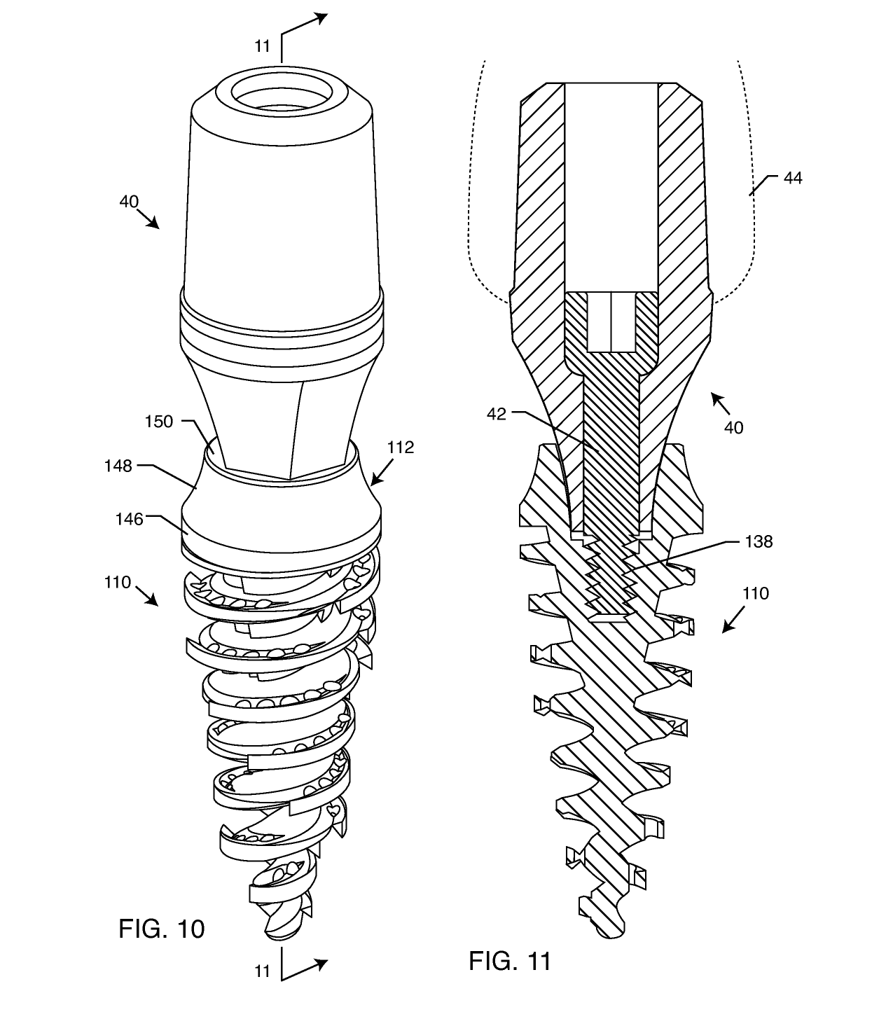

The bone implant 10 and 110 of the present invention is particularly suited for use as a dental implant. As such, the head 12 or 112 includes an internal connection 36 and 136 with internal threads 38 and 138 for an attachment of an abutment 40. Such abutments 40 are well known in the art. The abutment may be hollow, as illustrated in FIGS. 10 and 11, so as to receive a fastener 42, which engages the internal threads 38 or 138 so as to fasten the abutment 40 to the implant 10 or 110. With continuing reference to FIGS. 10 and 11, as is well known in the art, a false tooth or other prosthetic 44 is formed over the abutment 40, such as by firing ceramic material onto the abutment which mimics the patient's original tooth or teeth.

As illustrated in FIGS. 4 and 9, in a particularly preferred embodiment, the beveled internal connection is longer in the present invention for stability of the abutment 40. The longer internal bevel and shorter abutment cone connection provide stability for the abutment 40.

With reference now to FIG. 12, a prior art implant 2 is illustrated fastened to the jawbone 4 of the patient. The alveolar crest of the underlying bone 4 is often uneven. Thus, when the original tooth is removed and a typical prior art dental implant 2 installed, the gum tissue 6 can only grow straight up on the side of the implant 2 approximately two millimeters above the bone level. In many cases, a portion of the prior art implant remains exposed, as illustrated in FIG. 12.

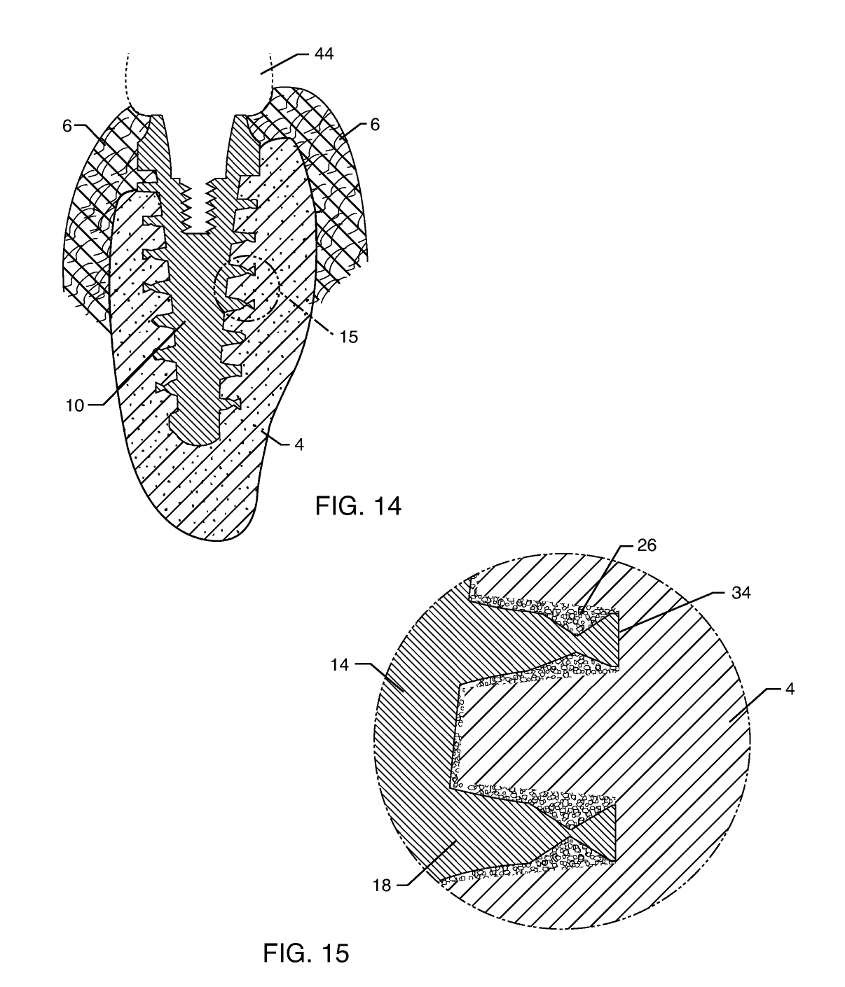

The head 12 design of the present invention overcomes this problem. The head 12 is generally comprised of a generally cylindrical neck portion 46. The portion 48 between the lower neck 46 and an upper surface 50 of the head 12 is generally cone-shaped, so as to be concave, as illustrated. Thus, the widest diameter of the neck 46 converges as a curve to the implant opening on the top surface 50. As such, the implant 10 is designed to be placed sub-crestal. This design allows taking advantage of the maximum alveolar crest bone available, as the neck 46 is circumferentially submerged in bone 4 while the top of the implant may be placed at or below the highest part of the cortical bone. This is a great advantage as it accommodates the invariable unevenness of the alveolar crest bone. Moreover, the gum tissue 6 is allowed to grow both straight up and over the neck 46 and curved portion 48, as illustrated in FIG. 14, such that there is no implant head exposure due to lack of coverage by the gum tissue 6.

Typically, the neck 46 is as wide as the upper-most blade 18. The implants may be offered in different diameters, such as narrow, regular and wide. The choice at each size depends upon the width of the alveolar crest of the area the implant is intended to be used. Thus, for example, the narrow size may be between 3-4 mm, the regular size 4-5 mm, and the wide 5-7 mm. Of course, the invention is not limited to such exact dimensions.

It will be appreciated by those skilled in the art that the implant of the present invention can be placed in locations which are otherwise not feasible with current implants and techniques. For example, sockets of freshly extracted multi-rooted teeth, where existing bone is very thin at the ideal position of the implant, the standard osteotomy can totally remove the existing bone making primary stabilization of the implant impossible.

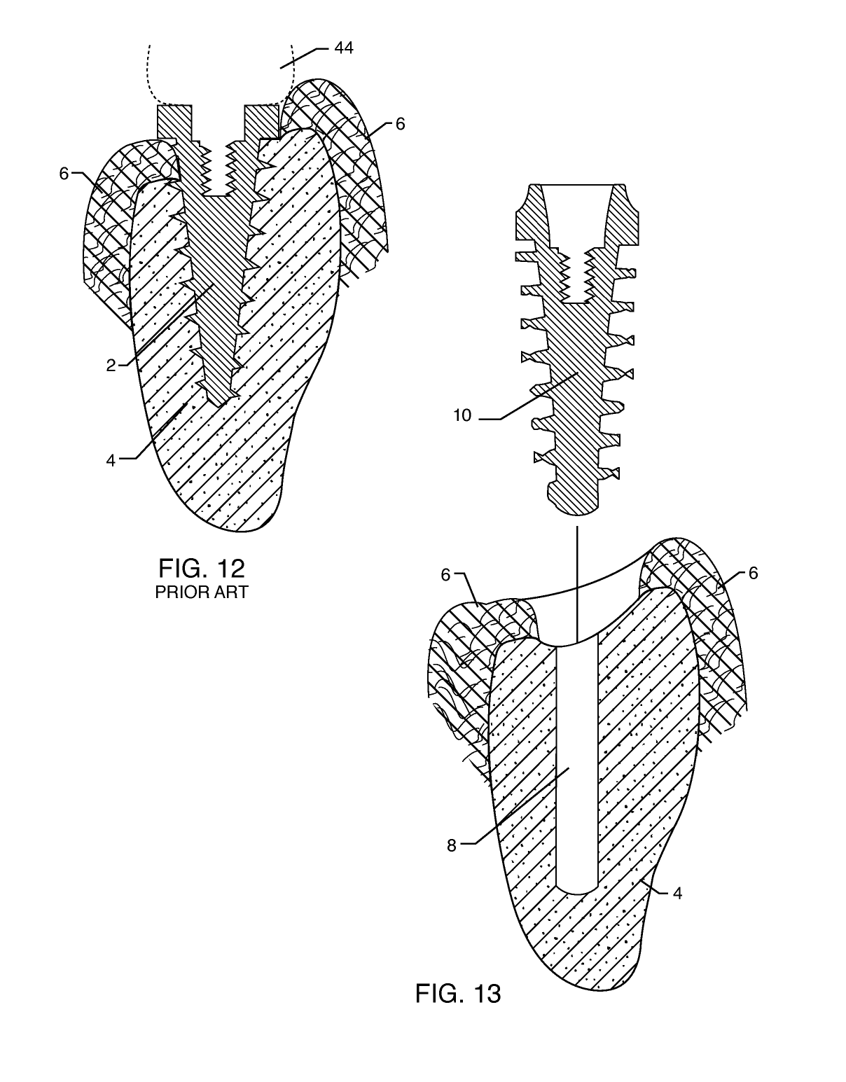

However, the implant of the present invention is only limited to the size of the pilot drill hole 8 (typically 1-2 mm) which corresponds with the diameter of the rounded tip 16 of the implant 10, as shown in FIG. 13. Thus, due to the fact that the implant 10 of the present invention is self-osteotomizing, it can be placed in sockets where prior art implants cannot be placed. Furthermore, collecting and using the bone shavings, as illustrated in FIG. 15, within the depressions 26 of the blades 18 allows the shavings to become excellent, vital autogenous bone grafts. Furthermore, the apertures 32 and 132 in the blades 18 allow collateral circulation to the bone between the blades 18 and 118.

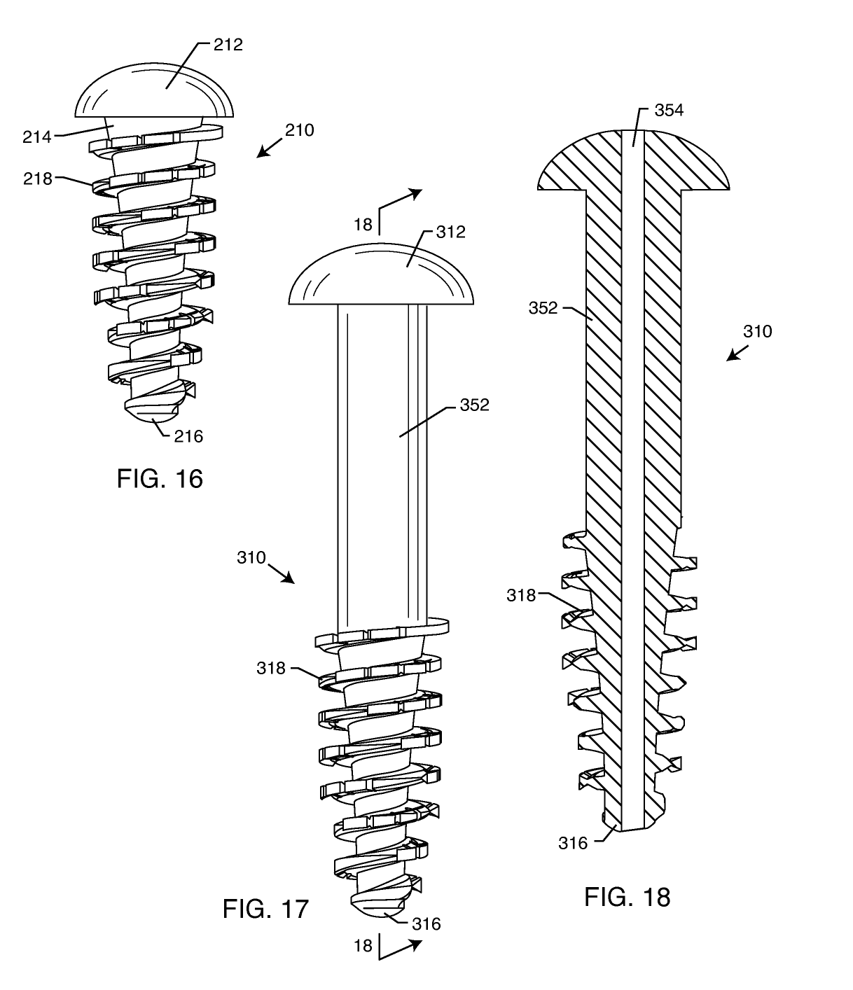

With reference now to FIGS. 16-18, the self-osteotomizing and grafting bone implant of the present invention is not necessarily limited to dental implants. Its features and advantages can be advantageously used in other bone implant/fastening circumstances. With particular reference to FIG. 16, a bone implant 210 or fastener is shown with a more traditional cone or flat head 212. Multiple osteotomy blades 218, having the features described above extend outwardly from a core body 214. Although the tip 216 is illustrated as being rounded, so as to be placed within a pilot hole, it is also conceived that in such instances the tip 216 could present a sharpened point so as to be driven into bone, such as during surgical operations such as those performed by orthopedists and the like, to fasten pieces of bone to one another, plates, devices and the like to bones, etc. It will be understood that the head 212 will have a slot or recess for a driver to drive the bone implant 210 into the bone.

With reference now to FIGS. 17 and 18, yet another bone implant 310 is illustrated for use in non-dental implant applications. In this case, there is an unthreaded portion 352 of the shaft between the head 312 and the tip 316. As such, the osteotomy blades 318 forming the spiral thread extend only partially along the core body 352 of the implant 310. This may be useful, for example, when attaching a plate or other device to a bone, wherein the lower portion containing the osteotomy blades 318 is inserted into the bone, and the non-threaded portion 352 extends through the plate, etc. FIG. 18 is a cross-sectional view of FIG. 16, taken generally along line 18-18, illustrating that a passageway 354 may be formed through the implant fixture 310 to serve the various purposes of the surgeon.

The non-dental applications of the bone implant of the present invention still experience the same advantages as the dental implant embodiments, in that the multiple osteotomy blades are responsible for gradual and unmatchable perfect osteotomy. The scooping feature of the individual blades due to the depressions formed within the blades preserve native bone and promote self-auto-grafting. Moreover, the apertures formed through the blades allow fluid and blood transfer between adjacent sections of bone and the bone shavings, and promotes the subsequent growth of blood vessels and new bone into the apertures. The stabilizing walls formed at the peripheral end of the osteotomy blades promote horizontal or lateral stabilization, as well as vertical or lineal stabilization.

Although several embodiments have been described in detail for purposes of illustration, various modifications may be made without departing from the scope and spirit of the invention. Accordingly, the invention is not to be limited, except as by the appended claims.

What is claimed is:

1. A self-osteotomizing and grafting bone implant, comprising:
a head;
a core body extending from the head to a tip and having a longitudinal axis; and
multiple osteotomy blades extending outwardly from at least a portion of the core body and arranged end to end forming a spiral thread;
wherein at least a portion of the osteotomy blades have an enlarged generally flat peripheral outer surface defining a stabilizing wall generally facing away from the core body.

2. The implant of claim 1, wherein the stabilizing wall defines a cutting edge.

3. The implant of claim 1, wherein an upper surface generally directed towards the head or a lower surface generally directed toward the tip of the osteotomy blades defines a depression adapted to channel bone material cut by the implant to a space between the upper and lower surfaces of adjacent osteotomy blades.

4. The implant of claim 3, wherein the depression is generally V-shaped or U-shaped.

5. The implant of claim 4, wherein the depression is defined by a first angled ramp of the surface of the osteotomy blade extending from the core body and a second angled ramp of the surface of the osteotomy blade extending from the peripheral outer surface of the osteotomy blade towards the first angled ramp.

6. The implant of claim 5, wherein at least a portion of the peripheral outer portion of the osteotomy blade has a generally triangular cross-section.

7. The implant of claim 1, wherein a plurality of osteotomy blades are end to end for each turn of the spiral thread.

8. The implant of claim 1, including a plurality of apertures formed through the osteotomy blades of a size and configuration permitting blood vessels and/or bone to grow therein.

9. The implant of claim 8, wherein surfaces and edges formed by the apertures are generally non-cutting flat or rounded edges and surfaces.

10. The implant of claim 8, wherein the apertures comprise open-face apertures formed in peripheral surfaces of the osteotomy blades.

11. The implant of claim 1, wherein the head of the implant comprises a dental implant head, wherein at least a portion of an outer surface of the dental implant head is generally concave.

12. The implant of claim 11, including a generally cylindrical neck adjacent the core body, and the generally concave outer surface of the head extending between the neck and an upper head surface.

13. The implant of claim 1, wherein the tip is rounded and has a diameter substantially matching a diameter of a pilot hole drilled into the bone.

14. The implant of claim 1, wherein at least a portion of the osteotomy blades adjacent the core body are of increasing cross-sectional thickness from the head towards the tip.

15. The implant of claim 1, wherein the implant is generally tapered from the head to the tip.

16. A self-osteotomizing and grafting bone implant, comprising:
a head;
a core body extending from the head to a tip and having a longitudinal axis; and
multiple osteotomy blades extending outwardly from at least a portion of the core body and arranged end to end forming a spiral thread;
wherein an upper surface generally directed towards the head or a lower surface generally directed toward the tip of the osteotomy blades defines a generally V-shaped or U-shaped depression adapted to channel bone material cut by the implant to a space between the upper and lower surfaces of adjacent osteotomy blades.

17. The implant of claim 16, wherein the depression is defined by a first angled ramp of the surface of the osteotomy blade extending from the core body and a second angled ramp of the surface of the osteotomy blade extending from the peripheral outer surface of the osteotomy blade towards the first angled ramp.

18. The implant of claim 16, wherein at least a portion of the osteotomy blades have an enlarged generally flat peripheral outer surface defining a stabilizing wall generally facing away from the core body and defining a cutting edge.

19. The implant of claim 18, wherein at least a portion of the peripheral outer portion of the osteotomy blade has a generally triangular cross-section.

20. The implant of claim 16, wherein a plurality of osteotomy blades are end to end for each turn of the spiral thread.

21. The implant of claim 16, including a plurality of apertures formed through the osteotomy blades of a size and configuration permitting blood vessels and/or bone to grow therein.

22. The implant of claim 21, wherein surfaces and edges formed by the apertures are generally non-cutting flat or rounded edges and surfaces.

23. The implant of claim 21, wherein the apertures comprise open-face apertures formed in peripheral surfaces of the osteotomy blades.

24. The implant of claim 16, wherein the head of the implant comprises a dental implant head, wherein at least a portion of an outer surface of the dental implant head is generally concave.

25. The implant of claim 24, including a generally cylindrical neck adjacent the core body, and the generally concave outer surface of the head extending between the neck and an upper head surface.

26. The implant of claim 16, wherein the tip is rounded and has a diameter substantially matching a diameter of a pilot hole drilled into the bone.

27. The implant of claim 16, wherein at least a portion of the osteotomy blades adjacent the core body are of increasing cross-sectional thickness from the head towards the tip.

28. The implant of claim 16, wherein the implant is generally tapered from the head to the tip.

29. A self-osteotomizing and grafting bone implant, comprising:
a head;
a core body extending from the head to a tip and having a longitudinal axis; and
multiple osteotomy blades extending outwardly from at least a portion of the core body and arranged end to end forming a spiral thread;
a plurality of apertures formed through the osteotomy blades of a size and configuration permitting blood vessels and/or bone to grow therein.

30. The implant of claim 29, wherein surfaces and edges formed by the apertures are generally non-cutting flat or rounded edges and surfaces.

31. The implant of claim 29, wherein the apertures comprise open-face apertures formed in peripheral surfaces of the osteotomy blades.

32. The implant of claim 29, wherein an upper surface generally directed towards the head or a lower surface generally directed toward the tip of the osteotomy blades defines a generally V-shaped or U-shaped depression adapted to channel bone material cut by the implant to a space between the upper and lower surfaces of adjacent osteotomy blades.

33. The implant of claim 32, wherein the depression is defined by a first angled ramp of the surface of the osteotomy blade extending from the core body and a second angled ramp of the surface of the osteotomy blade extending from the peripheral outer surface of the osteotomy blade towards the first angled ramp.

34. The implant of claim 29, wherein at least a portion of the osteotomy blades have an enlarged generally flat peripheral outer surface defining a stabilizing wall generally facing away from the core body and defining a cutting edge.

35. The implant of claim 34, wherein at least a portion of the peripheral outer portion of the osteotomy blade has a generally triangular cross-section.

36. The implant of claim 29, wherein a plurality of osteotomy blades are end to end for each turn of the spiral thread.

37. The implant of claim 29, wherein the head of the implant comprises a dental implant head, wherein at least a portion of an outer surface of the dental implant head is generally concave.

38. The implant of claim 37, including a generally cylindrical neck adjacent the core body, and the generally concave outer surface of the head extending between the neck and an upper head surface.

39. The implant of claim 29, wherein the tip is rounded and has a diameter substantially matching a diameter of a pilot hole drilled into the bone.

40. The implant of claim 29, wherein at least a portion of the osteotomy blades adjacent the core body are of increasing cross-sectional thickness from the head towards the tip.

41. The implant of claim 29, wherein the implant is generally tapered from the head to the tip.

42. A self-osteotomizing and grafting bone implant, comprising:
a head;
a core body extending from the head to a tip and having a longitudinal axis; and
multiple osteotomy blades extending outwardly from at least a portion of the core body and arranged end to end forming a spiral thread;
a plurality of apertures formed through the osteotomy blades of a size and configuration permitting blood vessels and/or bone to grow therein;
wherein an upper surface generally directed towards the head or a lower surface generally directed toward the tip of the osteotomy blades defines a generally V-shaped or U-shaped depression adapted to channel bone material cut by the implant to a space between the upper and lower surfaces of adjacent osteotomy blades; and
wherein at least a portion of the osteotomy blades have an enlarged generally flat peripheral outer surface defining a stabilizing wall generally facing away from the core body and defining a cutting edge.

43. The implant of claim 42, wherein surfaces and edges formed by the apertures are generally non-cutting flat or rounded edges and surfaces.

44. The implant of claim 42, wherein the apertures comprise open-face apertures formed in peripheral surfaces of the osteotomy blades.

45. The implant of claim 42, wherein the depression is defined by a first angled ramp of the surface of the osteotomy blade extending from the core body and a second angled ramp of the surface of the osteotomy blade extending from the peripheral outer surface of the osteotomy blade towards the first angled ramp.

46. The implant of claim 45, wherein at least a portion of the peripheral outer portion of the osteotomy blade has a generally triangular cross-section.

47. The implant of claim 42, wherein a plurality of osteotomy blades are end to end for each turn of the spiral thread.

48. The implant of claim 42, wherein the head of the implant comprises a dental implant head, wherein at least a portion of an outer surface of the dental implant head is generally concave.

49. The implant of claim 48, including a generally cylindrical neck adjacent the core body, and the generally concave outer surface of the head extending between the neck and an upper head surface.

50. The implant of claim 42, wherein the tip is rounded and has a diameter substantially matching a diameter of a pilot hole drilled into the bone.

51. The implant of claim 42, wherein at least a portion of the osteotomy blades adjacent the core body are of increasing cross-sectional thickness from the head towards the tip.

52. The implant of claim 42, wherein the implant is generally tapered from the head to the tip.

53. A self-osteotomizing and grafting dental implant bone implant, comprising:
a head having an internal connection for attachment of an abutment, an upper head surface and a lower head surface, the lower head surface being of a greater diameter than the upper head surface, and a neck extending between the upper and lower head surfaces, at least a portion of the neck defining a generally concave surface;
a core body extending from the head to a tip and having a longitudinal axis; and
multiple osteotomy blades extending outwardly from at least a portion of the core body and arranged end to end forming a spiral thread.

54. The implant of claim 53, wherein the neck portion has a generally cylindrical portion adjacent to the lower surface of the head.

55. The implant of claim 53, including a plurality of apertures formed through the osteotomy blades of a size and configuration permitting blood vessels and/or bone to grow therein.

56. The implant of claim 53, wherein at least a portion of the osteotomy blades has an enlarged generally flat peripheral outer surface defining a stabilizing wall generally facing away from the core body and defining a cutting edge.

57. The implant of claim 53, wherein an upper surface generally directed towards the head or a lower surface generally directed toward the tip of the osteotomy blades defines a generally V-shaped or U-shaped depression adapted to channel bone material cut by the implant to a space between the upper and lower surfaces of adjacent osteotomy blades.

58. The implant of claim 57, wherein the depression is defined by a first angled ramp of the surface of the osteotomy blade extending from the core body and a second angled ramp of the surface of the osteotomy blade extending from the peripheral outer surface of the osteotomy blade towards the first angled ramp.

59. The implant of claim 56, wherein at least a portion of the peripheral outer portion of the osteotomy blade has a generally triangular cross-section.

60. The implant of claim 53, wherein a plurality of osteotomy blades are end to end for each turn of the spiral thread.

61. The implant of claim 55, wherein surfaces and edges formed by the apertures are generally non-cutting flat or rounded edges and surfaces.

62. The implant of claim 55, wherein the apertures comprise open-face apertures formed in peripheral surfaces of the osteotomy blades.

63. The implant of claim 53, wherein the tip is rounded and has a diameter substantially matching a diameter of a pilot hole drilled into the bone.

64. The implant of claim 53, wherein at least a portion of the osteotomy blades adjacent the core body are of increasing cross-sectional thickness from the head towards the tip.

65. The implant of claim 53, wherein the implant is generally tapered from the head to the tip.
